(19) United States
(12) Patent Application Publication
(10) Pub. No.: US 2005/0131029 A1
(43) Pub. Date: Jun. 16, 2005

(54) Immunophilin ligand treatment of antiretroviral toxic neuropathy

(75) Inventors: Ahmet Hoke, Towson, MD (US); John W. Griffin, Hunt Valley, MD (US); Sanjay C. Keswani, Ellicott City, MD (US); Justin C. McArthur, Baltimore, MD (US)

Correspondence Address: EDWARDS & ANGELL, LLP, P.O. BOX 55874, BOSTON, MA, 02205, US

(73) Assignee: Johns Hopkins University, Baltimore, MD (US)

(21) Appl. No.: 10/836,517

(22) Filed: Apr. 30, 2004

Related U.S. Application Data
(60) Provisional application No. 60/466,650, filed on Apr. 30, 2003.

(51) Int. Cl. A61K 31/4439; G01N 33/53; G01N 33/567

(52) U.S. Cl. 514/343; 435/7.2

(57) ABSTRACT

The present invention relates to in vitro models of identifying non-immunosuppressive immunophilin ligands that are useful in the treatment or prevention of peripheral neuropathies. Other embodiments of the present invention include methods of using non-immunosuppressive immunophilin ligands for the treatment of antiretroviral toxic neuropathies.

### PRIORITY

The present application claims priority to U.S. Provisional Application No. 60/466,650, filed Apr. 30, 2003.

### FIELD OF THE INVENTION

The field of the invention relates to in vitro models for identifying agents that are useful in the treatment or prevention of human neuropathies.

### BACKGROUND OF THE INVENTION

HIV-associated sensory neuropathies are the most common neurological complications in HIV infection, clinically affecting about 30% of patients with AIDS. These include distal sensory polyneuropathy (DSP), associated with HIV infection per se, and antiretroviral toxic neuropathy (ATN), associated with Nucleoside Analogue Reverse Transcriptase Inhibitors (NRTIs), particularly ddC, ddl and d4T (reviewed in (Wulff et al., 2000)). Because the presently available symptomatic therapies are poorly efficacious the development of ATN often results in discontinuation of the offending drug in a particular patient. As these drugs are an essential component of highly active antiretroviral therapy (HAART) and substantially reduce the morbidity and mortality of HIV infection, this associated toxic sensory neuropathy not only affects quality of life, but also severely limits viral suppression strategies. Development of therapeutic drugs for treatment or prevention of ATN is hampered by the fact that there are no good established in vivo or in vitro models of NRTI-induced sensory neuropathy.

The immunophilins are a highly conserved group of chaperone proteins that are enriched in neurons of both the central and peripheral nervous system. As well as being immunosuppressants, the immunophilin ligands, FK506 and Cyclosporin A (CSA), also have neuroprotective and neurotrophic properties (reviewed in (Gold, 2000; Snyder et al., 1998)). Both these agents have been shown to be protective in vitro against glutamate neurotoxicity in cortical cultures (Dawson et al., 1993). Furthermore, in a recent study we showed that FK506 protected unmyelinated fibers of the cavernous nerve in an in vivo model of peripheral nerve injury sustained during radical prostatectomies (Sezen et al., 2001).

A drawback to the clinical use of FK506 as a neuroprotective agent is that it is also a potent immunosuppressant due to inhibition of calcineurin-dependent dephosphorylation of NFAT. This is a particularly major concern in the prevention of ATN in already immunocompromised HIV-positive patients. The development of in vitro models that identify non-immunosuppressive immunophilin ligands would be highly useful screening tools to test the efficacy of these agents in the treatment and prevention of ATN.

### SUMMARY OF THE INVENTION

The present invention relates to in vitro models of identifying non-immunosuppressive immunophilin ligands that are useful in the treatment or prevention of peripheral neuropathies. Other embodiments of the present invention include methods of using non-immunosuppressive immunophilin ligands for the treatment of antiretroviral toxic neuropathies.

A preferred embodiment of the present invention relates to a method of identifying immunophilin ligands useful for the treatment of antiretroviral toxic neuropathies comprising;
a) treating separate neuronal cell cultures with a NRTI, the same NRTI and an immunophilin ligand and vehicle control;
b) determining the mitochondrial toxicity in the separately treated cultures;
c) comparing the results on mitochondrial toxicity in the separately treated cultures;
d) identifying an immunophilin ligand which results in less mitochondrial toxicity due to the treatment with NRTI.

Another preferred embodiment of the present invention relates to a method of identifying immunophilin ligands useful for the treatment of antiretroviral toxic neuropathies comprising;
a) treating separate neuronal cell cultures with a NRTI, the same NRTI and an immunophilin ligand and vehicle control;
b) determining the effects on the number of neurites and total neuritic length per neuron in the separately treated cultures;
c) comparing the results on the number of neurites and total neuritic length per neuron in the separately treated cultures;
d) identifying an immunophilin ligand which results in preventing reduction in the number of neurites and total neuritic length per neuron due to the treatment with NRTI.

Still another preferred embodiment of the present invention relates to a method of identifying immunophilin ligands useful for the treatment of antiretroviral toxic neuropathies comprising;
a) treating separate neuronal cell cultures with a NRTI, the same NRTI and an immunophilin ligand and vehicle control;
b) determining the effects on neuronal cell death in the separately treated cultures;
c) comparing the results on neuronal cell death in the separately treated cultures;
d) identifying an immunophilin ligand which results in preventing neuronal cell death due to the treatment with NRTI.

Other emobodiments of the present invention include methods for the treatment or prevention of antitretroviral toxic neuropathy comprising the administration to a patient in need of such treatment or prevention a non-immunosuppressive immunophilin ligand.

Antiretroviral toxic neuropathy (ATN) is the most common neurological complication of HIV infection. This painful neuropathy not only affects the quality of life of HIV-infected patients but also severely limits viral suppression strategies. The present invention relates to in vitro models of this toxic neuropathy that mimics the in vivo situation in that ddC appears to be the most neurotoxic, followed by ddl and then d4T. AZT, which does not cause a peripheral neuropathy in patients, does not cause significant neurotoxicity in the models of the present invention. In these models, the immunophilin ligand FK506 prevents the development of neurotoxicity by ddC, as judged by amelioration of ddC-induced ‘neuritic pruning’, neuronal mitochondrial depolarization and neuronal necrotic death. The in vitro models of the present invention are highly useful screening tools to test the efficacy of promising neuroprotective or regenerative agents in the prevention or treatment of ATN.

### BRIEF DESCRIPTION OF THE FIGURES

FIG. 1 illustrate that NRTIs cause toxicity to primary DRG sensory neurons: examples of the changes seen in primary DRG sensory neurons in control (A), ddC at 1 μM (B), ddC at 10 μM (C) and ddC at 100 μM (D) treated cultures; also NRTIs associated with neuropathy resulted in dose dependent reduction in the number of neurites per neuron (E) and the total neuritic length per neuron (F).

FIG. 2 illustrates that FK506 but not CSA protects against ddC-induced neurotoxicity: DRG sensory neurons had long neurites in vehicle (A) and ddC+FK506 (C) treated cultures but not in ddC alone (B); FK506, but not CSA protected against ddC-induced reduction in the number of neurites (D) and total neuritic length (E) per neuron; (* ddC versus control P<0.05; ** ddC versus ddC+FK506P<0.05;)

FIG. 3 illustrates that ddC causes reduction in mitochondrial membrane potential differential (Δ-σ) and this is partially reversed by FK506: in vehicle-treated cultures (A-C) there is bright luminosity in the “red” wavelength; in FCCP (D-F) and ddC (G-1) treated cultures there is a reduction in the JC-1 fluorescence in the “red” wavelength; in ddC+FK506-treated cultures (J-L) many of the neurons have bright red fluorescence indicative of healthy mitochondria with intact Δ-σ; Panels A, D, G and J denote TTC labeling of the neurons in the field; Panels B, E, H, and K are JC-1 aggregate fluorescent emission in the “red” wavelength; Panels C, F, I and L are JC-1 monomer fluorescent emission in the “green” wavelength.

FIG. 4 illustrates that ddC, ddl and d4T, but not AZT cause a reduction in mitochondrial membrane potential differential (Δ-σ) and this is partially reversed by FK506: the JC-1 red:green fluorescence is expressed as a percentage of the vehicle-treated neurons in the same set of experiments; (* NRTI versus control P<0.05; ** ddC versus ddC+FK506P<0.05;).

FIG. 5 illustrates that ddC induces cell death that is not reversible by a caspase inhibitor, DVED, but is preventable by low doses of FK506; (* ddC versus control P<0.05; ** ddC versus ddC+FK506P<0.05;).

### DETAILED DESCRIPTION OF THE INVENTION

It is understood that this invention is not limited to the particular materials and methods described herein. It is also to be understood that the terminology used herein is for the purpose of describing particular embodiments and is not intended to limit the scope of the present invention which will be limited only by the appended claims. As used herein, the singular forms “a”, “an”, and “the” include plural reference unless the context clearly dictates otherwise. For example, a reference to “a neuronal cell” includes a plurality of such neuronal cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

In Vitro Models for Identifying Immunophilin Ligands for Treating ATN

The introduction of HAART regimens in 1996 has resulted in a dramatic improvement in the survival of HIV positive patients, with concomitant declines in the incidence rates of HIV-associated dementia and CNS opportunistic infections. HIV associated sensory neuropathies (DSP and ATN), by contrast, remain a very significant clinical problem and have emerged as the most common neurological complication in HIV infection. Not only do these painful neuropathies affect the quality of life of many HIV patients, but they also represent a major limiting factor on HAART viral suppression regimens that increasingly involve dideoxynucleoside agents

It has been hypothesized that NRTI toxicity is mediated by mitochondrial dysfunction. Complications of NRTI therapy resemble the clinical manifestations of inherited mitochondrial diseases. Examples include hepatic steatosis, lactic acidosis, ‘ragged red fiber’ myopathy (AZT), pancreatitis (ddl; D4T) as well as peripheral neuropathy. Furthermore, mitochondrial ultrastructural abnormalities have been noted in affected tissues (Lewis and Dalakas, 1995). An increased serum lactate level and a reduced serum acetyl-carnitine level have been observed in patients with ATN (Brew et al., 2001; Famularo et al., 1997). It also has been demonstrated that there are reduced levels of mitochondrial DNA in subcutaneous tissues with dideoxynucleoside usage (Cherry et al., 2002 In press). As all the NRTIs have been shown in vitro to inhibit mitochondrial DNA polymerase-γ, the host enzyme responsible for mitochondrial DNA synthesis, it has been hypothesized that mitochondrial toxicity may result from mitochondrial DNA depletion (Starnes and Cheng, 1987). However, in a study by Cui and coworkers, although ddl and ddC toxicity on PC-12 cells correlated with inhibition of mitochondrial DNA synthesis, no change in mitochondrial DNA content was observed by d4T at doses that caused toxic effects on neurites (Cui et al., 1997). This suggests that NRTI mitochondrial toxicity may be mediated by mechanisms other than mitochondrial DNA depletion.

To directly assess the presence of mitochondria dysfunction at pharmacologically relevant doses of NRTIs that cause neurotoxicity, JC-1 was used, a fluorescent lipophilic cationic dye that accumulates in mitochondria in proportion to the electrical potential differential (Δ-σ) that normally exists across the inner mitochondrial membrane (Cossarizza et al., 1993; Reers et al., 1991). This technique has been used successfully to assess the functional status of mitochondria in cultured neurons (White and Reynolds, 1996) and cardiomyocytes (Mathur et al., 2000). As mixed DRG cultures were used, Tetanus toxin C-fragment (TTC) also was used as a live neuronal marker. This is known to be a retrogradely transported neuronal tracer that binds to ganglioside GT1b (Lazarovici et al., 1987) and is taken up by most peripheral neurons (Stockel et al., 1975). The results showed that after 4 hours of incubation, ddC, ddl and d4T resulted in dose-dependent neuronal mitochondrial membrane depolarization. Correlating with the morphological neurotoxicity data, ddC was the most potent in causing mitochondrial toxicity, followed by ddl and then d4T. Furthermore, AZT did not cause significant neuronal mitochondrial depolarization, even at high doses (100 μM).

Due to the rapidity of the observed neurotoxicity in the models of the present invention by the NRTIs, it is doubtful that mitochondrial DNA depletion, by DNA polymerase-γ inhibition, is responsible for the observed effect. It has been estimated that significant mitochondrial DNA depletion caused by the NRTIs takes place only after several days to weeks of exposure (Chen and Cheng, 1989; Chen et al., 1991; Lewis et al., 1992). Observations in human subcutaneous fat suggests that mtDNA may normalize after prolonged exposure to dideoxynucleosides and that the inhibition of DNA polymerase-γ is potentially reversible (Cherry et al., 2002 In press). Moreover, human cell lines (termed ρ0) completely depleted of mitochondrial DNA by repeated ethidium bromide incubation have normal mitochondrial membrane potentials (Jiang et al., 1999). Chen and colleagues did not find any correlation between the ability of nucleoside analogs to increase lactate production and their potency in mtDNA depletion (Chen et al., 1991). It is more likely that the NRTIs exert rapid toxicity by directly inhibiting mitochondrial bioenergetic function in a tissue-specific fashion. Indeed, AZT has been shown to inhibit NADH-linked respiration and NADH-cytochrome c reductase activity in isolated rat skeletal muscle, brain and liver mitochondria (Modica-Napolitano, 1993). Furthermore, AZT inhibits adenylate kinase and the ADP/ATP translocator in isolated liver mitochondria, resulting in early impairment of oxidative phosphorylation (Barile et al., 1994; Barile et al., 1997; Hobbs et al., 1995). In another study (Benbrik et al., 1997), AZT was shown, in cultured human muscle cells, to reduce the activity of SDH, a complex II protein that is encoded by nuclear rather than mitochondrial DNA. Skuta and colleagues noted that ddC induced rapid cardiotoxicity in rats, and this was associated with decreased activity of respiratory complexes, but not with mitochondrial DNA depletion (Skuta et al., 1999).

The immunophilin ligands, FK506 and CSA have been demonstrated to be neuroprotective in in vitro excitotoxicity paradigms and in vivo stroke models (Dawson et at., 1993; Sharkey and Butcher, 1994; Toung et at., 1999). However, the molecular mechanisms governing this neuroprotection are unknown. Some studies have indicated that neuroprotection is via inhibition of calcineurin, a calcium-dependent protein phosphatase, that has among its substrates neuronal NO synthase and BAD, which have both been implicated in death pathways (Dawson et at., 1993; Wang et al., 1999). CSA, in addition, has been shown to inhibit the mitochondrial permeability transition (mPT) pore, which is thought to be a key player in excitotoxic neuronal death (Ankarcrona et at., 1996; Crompton et at., 1998; Dubinsky and Levi, 1998). In our in vitro model of ATN, FK506 in the nanomolar range potently prevented toxicity by ddC on primary dorsal root ganglion neurons. It significantly ameliorated ddC-induced neuritic ‘pruning’, neuronal mitochondrial depolarization at 4 hours; and neuronal necrotic death at 18 hours.

In vitro models of ATN of the present invention show that NRTIs cause neurotoxicity on primary DRG sensory neurons in a manner that reflects their potency in causing neuropathy in HIV-infected individuals. Furthermore, when FK506 was administered to the DRG cultures at the time of addition of ddC, this neurotoxicity was prevented.

In the in vitro models of NRTI toxicity in primary DRG neuronal cultures, two separate morphological parameters were used (number of neurites and total neuritic length per neuron) to measure neurotoxicity. These models show that ddC, ddl and d4T, in a dose-dependent manner, inhibit further elongation of neuritic length in primary sensory neuronal cultures. Furthermore, these drugs cause a notable ‘pruning’ of established neurites, with decreased neuritic number and branch points per neuron. Also seen, even at low doses of NRTI, was segmental neuritic ‘beading’ particularly prominent in the most distal portions of the neurites, away from the cell body. These varicosities can be seen in early Wallerian-like degeneration of axonal processes (Fayaz and Tator, 2000). Indeed at higher doses of ddC, frank neuritic degeneration was seen.

The in vitro neurotoxicity data suggested that the potency of each drug in inhibiting neuritic outgrowth paralleled the potency of each drug in causing peripheral neuropathy in humans. In experiments, ddC was the most neurotoxic followed by ddl, and d4T was the least toxic. This profile is very similar to the incidence of neuropathy in HIV infected patients who are on these drugs (ddC>ddl>d4T) (Wulff et al., 2000). AZT, which does not cause a peripheral neuropathy in patients, did not exert significant neurotoxicity in the present invention models. Furthermore, the doses used in the study are pharmacologically relevant doses (Benbrik et al., 1997). Consequently, the present invention in vitro models of NRTI neurotoxicity are clinically applicable as models of ATN, and represent helpful tools to evaluate mechanisms of NRTI neurotoxicity and to screen agents for neuroprotective efficacy.

A preferred embodiment of the present invention relates to a method of identifying immunophilin ligands useful for the treatment of antiretroviral toxic neuropathies comprising;
a) treating separate neuronal cell cultures with a NRTI, the same NRTI and an immunophilin ligand and vehicle control;
b) determining the mitochondrial toxicity in the separately treated cultures;
c) comparing the results on mitochondrial toxicity in the separately treated cultures;
d) identifying an immunophilin ligand which results in less mitochondrial toxicity due to the treatment with NRTI.

Another preferred embodiment of the present invention relates to a method of identifying immunophilin ligands useful for the treatment of antiretroviral toxic neuropathies comprising;
a) treating separate neuronal cell cultures with a NRTI, the same NRTI and an immunophilin ligand and vehicle control;
b) determining the effects on the number of neurites and total neuritic length per neuron in the separately treated cultures;
c) comparing the results on the number of neurites and total neuritic length per neuron in the separately treated cultures;
d) identifying an immunophilin ligand which results in preventing reduction in the number of neurites and total neuritic length per neuron due to the treatment with NRTI.

Still another preferred embodiment of the present invention relates to a method of identifying immunophilin ligands useful for the treatment of antiretroviral toxic neuropathies comprising;
a) treating separate neuronal cell cultures with a NRTI, the same NRTI and an immunophilin ligand and vehicle control;
b) determining the effects on neuronal cell death in the separately treated cultures;
c) comparing the results on neuronal cell death in the separately treated cultures;
d) identifying an immunophilin ligand which results in preventing neuronal cell death due to the treatment with NRTI.
   Cells used in the Methods of the Invention:

Neural cells useful in the methods of this invention can be used after isolation from a donor or donors or may be obtained from in vitro culture. Preferably, the cells of the invention are of mammalian origin, i.e., are obtained from mammalian subjects (e.g., humans, mice, or rats). Preferred cells for use in the instant methods are rodent cells. Other preferred cells are human. Neural cells obtained form human fetal tissues can be used in these assays in the same manner as the rodent neural cells.

Neural cells useful in the methods of this invention may be obtained during various stages of development of the donor subject, e.g., can be embryonic fetal, juvenile, or adult cells. In general, the particular stage of development is selected based upon the intended use of the cells subsequent to storage and the species of animal from which the cells are derived.

Preferably, neural cells can be obtained from the dorsal root ganglion.

In other embodiments, the cells for use in the instant methods are neural precursor cells. In one embodiment, the neural stem or progenitor cells are induced to differentiate into dorsal root ganglion neurons for use in the assays. Tissue containing stem or progenitor cells can be obtained from mammalian embryos, fetuses, juveniles, or from an adult organ donor. In preferred embodiments, stem cells to be used in the instant methods are rodent cells. In other preferred embodiments, stem cells to be used in the instant methods are human cells.

Neural precursor cells can be obtained from any area of the central nervous system, including the cerebral cortex, cerebellum, midbrain, brainstem, spinal cord, ventricular tissue, or from areas of the peripheral nervous system, including the dorsal route ganglion, the carotid body and the adrenal medulla. Methods of obtaining neural progenitor or stem cells are known in the art (see e.g., U.S. Pat. No. 5,753,506; WO97/44442; WO96/04368; WO94/10292; WO94/02593; Gage et al. 1995 Ann. Rev. Neurosci. 18:159; or WO98/30678).

To expand a population of neural cells, (e.g., stem or progenitor cells) the cells can be grown in the presence of trophic factors, such as nerve growth factor, acidic fibroblast growth factor, basic fibroblast growth factor, platelet-derived growth factor, thyrotropin releasing hormone, epidermal growth factor, amphiregulin, transforming growth factor, transforming growth factor beta., insulin-like growth factor, or other growth factors using methods known in the art (see, e.g., U.S. Pat. Nos. 5,753,506, 5,612,211, 5,512,661, WO93/01275; Mehler and Kessler. 1995 Crit. Rev. Neurobiol. 9:419; and WO 98/30678).

In other embodiments, neural stem cells can be induced to differentiate using agents which are known in the art, e.g., retinoic acid, butyrate, triodo-thyronine, or s-laminin.

In one embodiment, the cells for use in the present invention are fetal or embryonic cells. Preferably, the cells are derived from the fetal central nervous system. In another embodiment, the fetal cells are spinal cord cells. In preferred embodiments, the fetal cells are dorsal root ganglion cellsThe cells described herein can be grown as a cell culture, i.e., as a population of cells which grow in vitro, in a medium suitable to support the culture (e.g., growth or stimulation) of the cells prior to administration to a subject. Media which can be used to support the growth of neural cells include mammalian cell culture media, such as those produced by Gibco BRL (Gaithersburg, Md.). See 1994 Gibco BRL Catalogue & Reference Guide. In addition, other substrates upon which the neural cells can grow including, for example, collagen, collagen plus poly-omithine and poly-omithine plus fibronectin, can be used. The medium can be serum-free or supplemented with animal serum such as fetal calf serum. Moreover, growth factors, e.g., neurotrophic factors, can be added to the cell culture to promote neural cell growth in vitro. Examples of neurotrophic factors include glial cell line-derived growth factor, brain-derived neurotrophic factor, platelet-derived growth factor, neural growth factor, ciliary neurotrophic factor, midkine, insulin-like growth factor I and II, insulin, fibroblast growth factor, neurotrophin-3, neurotrophin 4/5 and transforming growth factor beta.

Methods of Treating or Preventing Antiretroviral Toxic Neuropathies Immunophilin Ligands:

Immunophilins are specific high-affinity receptors for immunosuppressant drugs. These include cyclophilin and FKBP, which bind to cyclosporin A, and FK-506, respectively. In addition to these compounds, other immunophilin-binding drugs have been developed. Such drugs include rapamycin, FK-520, FK-523, 15-0-DeMe-FK-520, (4R)-)(E)-L-butenyl 1-4, N-dimethyl-L-threonine.

The neurotrophic actions of immunophilin ligands have therapeutic ramifications. The extreme potency of FK506 is in the range of neurotrophic proteins. Drugs such as FK506 are readily synthesized and can cross the blood brain barrier. Thus, besides therapeutic effects for neuroprotection in conditions such as stroke, FK506 and other small molecules that interact with immunophilins are of use in facilitating neuronal repair. Situations where neuronal repair can be facilitated include, but are not limited to diseases including peripheral nerve damage, whether by physical injury or disease state such as diabetes. In addition, facilitation of neuronal repair is useful for injury or disease states of the central nervous system (spinal cord and brain) including physical damage to the spinal cord, damage to motor neurons such as occurs in amyotrophic lateral sclerosis, brain damage as occurs in strokes, Alzheimer's disease and Parkinson's disease.

The immunophilin ligands useful in the methods of the present invention are non-immunosuppressive and can be determined as such by methods, including but not limited to, those described below.

The dosage and length of treatment with non-immunosuppressive immunophilin-binding drugs depends on the disease state of the ATN. The duration of treatment may be a day, a week, or longer, and may, in the case of a chronic progressive illness, last for decades. The non-immunosuppressive immunophilin-binding drugs are administered in a therapeutically effective amount, a typical human dosage of a FK-506 analog that is non-immunosuppressive, for example, ranging from about 0.1 mg/kg of body weight of the FK-506 analog to about 1.0 mg/kg of FK-506, in single or divided doses. The dosage will vary depending on the non-immunosuppressive immunophilin-binding drug to be used and its relative potency. Dosage and length of treatment are readily determinable by the skilled practitioner based on the condition and stage of disease.

As used herein, the term “FK506 analogs” refers to compounds that are functionally analogous to FK506 in their ability to stimulate neuritic outgrowth, but also are non-immunosuppressive. Such FK506 analogs, such as V-10,367, retain the FKBP12 binding domain but lack the structural components of the effector domain and may either bind FKBP12 or be non-binding. V-10,367, for example, binds FKBP12 with high affinity (<1 nM) (Armistead et al., Acta Crystallogr. 51:522-528, 1995).

There has been an intense effort to design compounds that are structurally related to FK506. See, for example: Bierer et al., Science 250:556-559, 1990; Van Duyne et al., Science 252:839-842, 1991; Van Duyne et al., J. Mol. Biol. 229:105-124, 1993; Hauske et al., J. Med. Chem. 35:4284-4296, 1992; Holt et al., J. Am. Chem. Soc. 115:9925-9938, 1993; Holt et al., Bioorg. Med. Chem. Lett. 3:1977-1980, 1993; Teague and Stocks, Bioorg. Med. Chem. Lett. 3:1947-1950, 1993; Wang et al., Bioorg. Med. Chem. Lett. 4:1161-1166, 1994; Yamashita et al., Bioorg. Med. Chem. Lett. 4:325-328, 1994; Stocks et al., Bioorg. Med. Chem. Lett. 4:1457-1460, 1994; Goulet et al., Perspect. Drug Disc. Design 2:145-162, 1994; Wilson et al., Acta Cryst. D51:511-S21, 1995; Armistead et al., Acta Cryst. D51:522-528, 1995; U.S. Pat. Nos. 5,192,773, 5,330,993, 5,516,797, 5,612,350, 5,614,547, 5,622,970, 5,654,332; and published international patent applications WO 92/00278, WO 92/04370, WO 92/19593, WO 92/21313, WO 94/07858, and WO 96/40633.

FK506 analogs include, but are not limited to compounds disclosed in U.S. Pat. Nos. 5,622,970, 5,516,797, 5,330,993, 5,192,773, and WO 92/00278, and also for methods relating to the synthesis of these compounds, the disclosures of which are incorporated herein by reference.

Assays for Human FKBP Binding:

FK506 is known to bind to the human protein, FKBP12, and to form a tripartite complex with hFKBP12 and FRAP, a human counterpart to the yeast proteins TOR1 and TOR2. Analogs may be characterized and compared to FK506 with respect to their ability to bind to human FKBP12 and/or to form tripartite complexes with human FKBP12 and human FRAP (or fusion proteins or fragments containing its FRB domain). See WO 96/41865, the full contents of which are incorporated herein by reference. That application discloses various materials and methods which can be used to quantify the ability of a compound to bind to human FKBP12 or to form a tripartite complex with (i.e., “heterodimerize”) proteins comprising human FKBP12 and the FRB domain of human FRAP, respectively. Such assays include fluorescence polarization assays to measure binding. Also included are cell based transcription assays in which the ability of a compound to form the tripartite complex is measured indirectly by correlation with the observed level of reporter gene product produced by engineered mammalian cells in the presence of the compound. Corresponding cell-based assays may also be conducted in engineered yeast cells. See e.g. WO 95/33052 (Berlin et al).

Human Immunosuppression Assay Methods:

Assays for immunosuppressive activity are known. By way of non-limiting example, immunosuppressive activity (as distinguished from binding activities) may be measured in a mitogenesis assay using human T cells. Such a human T cell proliferation assay is an example of an appropriate in vitro assay for use in determining an immunosuppressive value for a analog of interest.

In one embodiment, a representative compound can be evaluated in an in vivo test procedure designed to determine the survival time of pinch skin graft from male BALB/c donors transplanted to male C3H(H-2K) recipients. The method is adapted from Billingham et al. (1951) J. Exp. Biol. 28:385-402. Briefly, a pinch skin graft from the donor is grafted on the dorsum of the recipient as an allograft, and an isograft is used as control in the same region. The recipients are treated with either varying concentrations of a test analog compound intraperitoneally, intravenously or orally. FK506 can be used as a test control. Untreated recipients serve as rejection control. The graft is monitored daily and observations are recorded until the graft becomes dry and forms a blackened scab. This is considered as the rejection day. The mean graft survival time (number of days+/−S.D.) of the drug treatment group is compared with the control group. An ED50 value can be calculated as the mean ratio of weight analog to weight animal required to produce a mean graft survival time extending to the same period as control graft. The immunosuppressant activities, e.g., immunosuppressant ED50 values of the analog compounds of this invention can be determined via the hemolysin test in mice and by the delayed hypersensitivity test

An exemplary hemolysin test is that described in Methods in Immunology, edited by D. H. Campbell et al, W. A. Benjamin, New York 1963 pages 172-175, and measures humoral or antibody response. The delayed hypersensitivity test measures the effect of a test analog compound on the ability of a subject mouse to mount a cell-mediated immune response to the antigen, Mycobacterium tuberculosis H37Ra. The mouse is sensitized to the antigen by subcutaneous administration in the base of the tail. The development of the delayed hypersensitivity response may be measured at any time beginning six days after sensitization but is usually done on the ninth day as follows: The right hind paw is injected with analog while the left hind paw (control) receives physiological saline. Both paw volumes are measured after twenty-four hours and significant increase in the volume of the right hind paw is taken as a measure of an effective delayed hypersensitivity response. All compounds are administered by the subcutaneous route. The expression ED50 (mg./kg.) is an expression of the number of milligrams of the analog per kilogram of body weight administered subcutaneously required to reduce the antibody activity by 50% when compared with a control. In this case, the higher the ED50 value for a analog the less potent an immunosuppressant it is, and as such, analogs with very high ED50 values can be considered non-immunosuppressive.

The immunosuppressive activity of a analog may also be shown by a graft vs. host reaction (GVHR). In an illustrative embodiment, to induce a GVHR, C57 B1/6XA/J(F6AF1) male mice are injected intravenously with parental (C57B1/6J) spleen and lymph node cells. The compound (a FK506 analog) is then administered orally for 10 days beginning on the day prior to the cell transfer. On the day following the last treatment, the animals are sacrificed, and their spleens excised and weighed. The enlargement of the spleen of the host is a result of a GVHR. To some extent it is the host's own cells which infiltrate and enlarge the spleen although they do this because of the presence of graft cells reacting against the host. The amount of spleen enlargement, splenomegaly, is taken as a measure of the severity of the GVHR. In carrying out the GVHR the animal in the experimental group is injected with parental cells, cells of the same species but of different genotype, which cause a weight increase of the spleen. The animal in the control group is injected with syngeneic cells, genetically identical cells which do not cause a weight increase of the spleen. The effectiveness (ED50) of the compounds administered to the mice in the experimental group is measured by comparing the spleen weight of the untreated and treated GVH animal with that of the syngeneic control. The ED50 value for immunosuppressive activity of analogs can also be measured according to the method of Takatsy et al. (1955) Acta. Microbiol. Acad. Sci. Hung., 3:105 or Cottney et at, (1980) Agents and Actions, 10:43.

The immunosuppressive activity of a FK506 analog may also be shown by a splenic atrophy test, e.g., a decrease in spleen weight after dosing BDF1 mice orally with the drug for seven (7) consecutive days. The mice are sacrificed on the eighth day. The percent decrease in spleen weight is measured for each dosage level.

Pharmaceutical Compositions:

Another aspect of the invention pertains to pharmaceutical compositions of immunophilin ligands useful in the methods of the invention. The pharmaceutical compositions of the invention typically comprise a compound useful in the methods of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Depending on the route of administration, the compound may be coated in a material to protect it from the action of enzymes, acids and other natural conditions which may inactivate the agent. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes (Strejan, et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., one or more immunophilin ligands) preferably is formulated in the composition in a therapeutically effective amount. A “therapeutically effective amount” refers to an amount effective, at dosages and for periods of time necessary, to achieve the desired therapeutic result to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

A compound of the invention can be formulated into a pharmaceutical composition wherein the compound is the only active agent therein. Alternatively, the pharmaceutical composition can contain additional active agents. For example, two or more compounds of the invention may be used in combination.

This invention is further illustrated by the following examples which should not be construed as limiting. The contents of all references, patents, and published patent applications cited throughout this application, as well as the figures, are incorporated herein by reference.

### EXEMPLIFICATION

The following methods and materials were used in the following Examples:

Preparation of Schwann Cell Cultures

Schwann cells were prepared from 1-day-old Sprague-Dawley rat pups and purified by a modified Brocke's method (Brockes et al., 1979). Two days prior to co-culture experiments, purified Schwann cells were dissociated using brief trypsinization and plated onto poly-L lysine and rat tail collagen (Collaborative Biomedical Products, Bedford, Mass.) coated glass coverslips in 24-well tissue culture plates at a density of 20,000 cells/well.

Preparation of DRG Neuronal Cell Cultures

Dissociated primary DRG neuronal cell cultures were prepared as described by Eldridge et al. (Eldridge et al., 1987). Briefly, the DRGs from day 15 embryos were dissected and dissociated with 0.25% trypsin in L-15 medium. Dissociated cells (13,000 cells/well) were then directly plated onto glass coverslips, already bearing a Schwann cell monolayer. Co-cultures were maintained in Neurobasal medium containing 1% fetal bovine serum (FBS) (HyClone, Logan, Utah) and GDNF (10 μg/ml).

Morphological Neurotoxicity and Neuroprotection Assays:

In this assay, the effect of NRTIs on established neuritic processes was.measured. DRG neurons were prepared as above, plated on Schwann cell monolayers and allowed to extend neurites for 3 hours. Varying concentrations of ddC, ddl, d4T and AZT (1-100 μM) or vehicle control were added at 3 hours. After 15 hours of further incubation, the cells were fixed and then immunostained with anti-βIII-tubulin antibody (Promega, Madison, Wis.) at 1:5000 dilution. In neuroprotection assays various doses of FK506 (1 nM, 10 nM, 100 nM, 1 μM) and CSA (10 nM, 100 nM, 1 μM, 10 μM) were added to the cultures at the same time as addition of ddC (10 μM) i.e. at 3 hours. Cells were then incubated for another 15 hours and stained as above.

Using confocal microscopy, the neuritic length of minimum 10 neurons per coverslip per condition was measured by unbiased stereological methods using an image analysis system on the confocal microscope. The total length of neurites and the number of neurites per neuron, were measured. Each experimental condition was done in triplicate wells and repeated 3-6 times. The results from each set of experiments were averaged and counted as n=1 for statistical analysis. Statistical significance was determined in Statview (Macintosh version 5.0.1) using ANOVA with correction for multiple comparisons (the critical alpha level set at p=0.005). Same method was used for the statistical analysis of other data (see below).

Neuronal Mitochondrial Toxicity Assays

Primary DRG sensory neuronal cultures were set up as above. Varying concentrations of ddC, ddl, d4T and AZT (1-100 μM), FCCP (a prototypic mitochondrial membrane depolarizer) (25 μM) or vehicle control were added at the time of plating. In neuroprotection assays FK506 (100 nM) or CSA (100 nM) were used along with ddC (10 μM). Tetanus toxin C-fragment (TTC) (List Biological Laboratories Inc.) was used as a live neuron-specific label and was added to the cultures 2 hours after plating at a final concentration of 0.4 g/ml. After one hour of incubation, the wells were washed with warm media, and a polyclonal anti-TTC antibody (1:5000 dilution) (Biogenesis, Kingston, N.H.) was added into the media for 30 minutes at 37° C. This was followed by washing in media and incubation in Alexa 633-conjugated rabbit anti-goat secondary antibody (1:200 dilution) (Molecular Probes, Eugene, Oreg.) for 30 minutes at 37° C. Four hours after plating, JC-1 (2.5 μg/ml) was added to the cultures according to the manufacturer's recommendations and published reports using JC-1 in central nervous system neurons (Ankarcrona et al., 1995; White and Reynolds, 1996). After incubation for 20-30 minutes at 37° C., the coverslips were washed with warm PBS. The fluorescence emission pattern of JC-1 in TTC-labeled cells was then immediately observed under confocal microscopy in both the red and green portions of the spectrum, and the red:green fluorescence ratio was calculated in a minimum of 20 neurons per coverslip.

Cell Death Assay

Primary DRG sensory neuronal cultures were set up as above. Varying concentrations of ddC, vehicle control or 100 μg/ml staurosporine (Sigma, St. Louis, Mo.) were added at the time of plating. In neuroprotection assays at the time of neuronal plating, ddC (10 μM), staurosporine (100 μg/ml) or vehicle control was added to the cultures, with or without DVED (10 μM) and varying doses of FK506 (1 nM, 10 nM, 100 nM, 1 μM) or CSA (10 nM, 100 nM, 1 μM, 10 μM). As described above for JC-1 analysis, TTC uptake was utilized for neuronal identification purposes. After 18 hours of incubation, the cells were stained with Ethidium homodimer and Calcein ester, according to methods detailed by the manufacturer's kit (Live/Death Kit, Molecular Probes, Eugene, Oreg.). Using unbiased stereological methods, the live neurons were counted and expressed as a percentage of all TTC-labeled cells (minimum 200 cells per group). In order to determine if the cell death was primarily apoptotic or necrotic, after 18 hours of incubation with ddC (10 μM) or vehicle control +/−DEVD (10 μM), the cells were fixed and stained with TUNEL, according to methods detailed by the manufacturer's kit (Sigma, St. Louis, Mo.). In order to identify neurons within the cultures, the cells were also immunostained with anti-βIII-tubulin antibody. Using confocal microscopy, TUNEL-positive neurons were identified, counted and expressed as a percentage of all neurons ie all βIII-tubulin-staining cells (minimum 200 cells per group).

### EXAMPLE 1

### NRTIs Cause Dose-Dependent Neurotoxicity on Primary DRG Sensory Neuronal Cultures

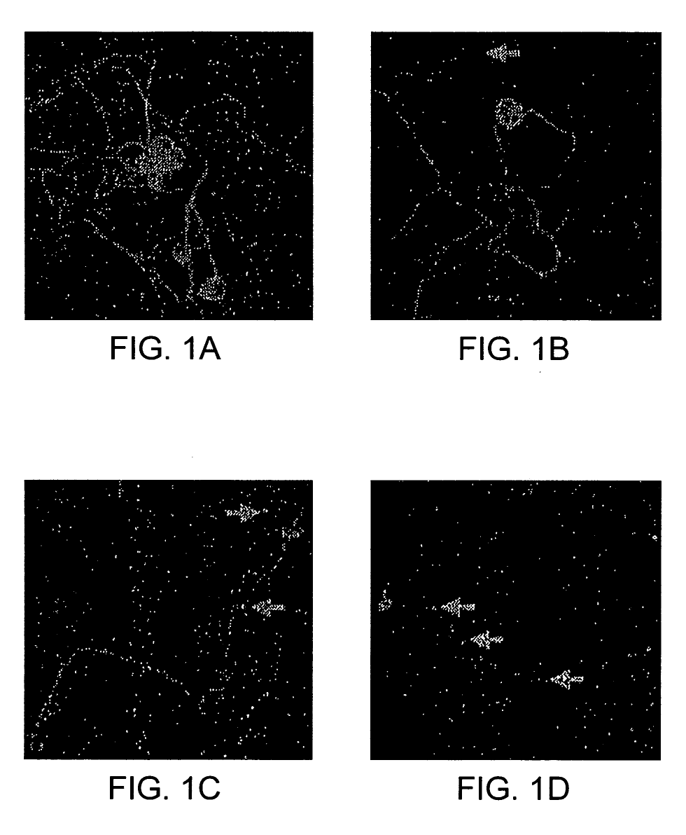

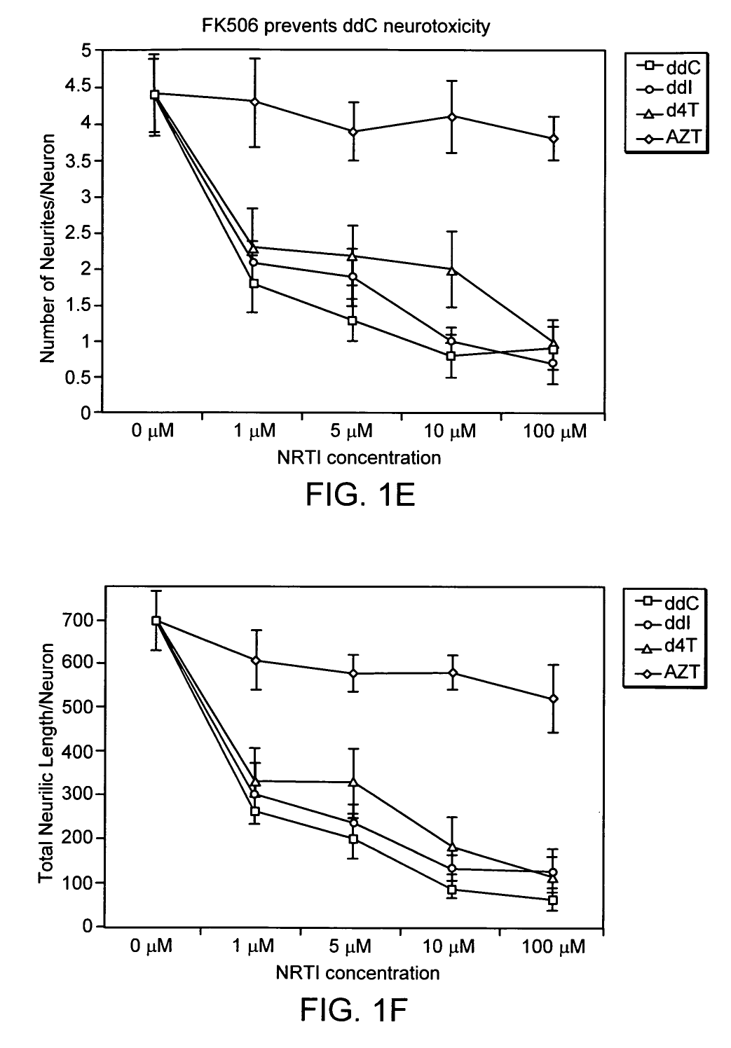

In order to assess the effects of NRTIs on established neurites primary DRG neurons were plated on a Schwann cell monolayer and allowed to extend their neurites for about 3 hours in the presence of GDNF-containing medium. At this early stage, most neurons had neurites that were at least 100 microns in length. Then added varying doses of NRTIs or vehicle control were added, before fixing and immunostaining the cultures 15 hours later. FIG. 1 (A-D) shows representative confocal microscope fields from a vehicle control culture and cultures treated with varying doses of ddC. In the control cultures, most neurons bore several neurites that were at least 200-300 microns in length and had many branching points. Morphological dose-dependent changes were noted in the ddC-treated neurons. At low ddC concentrations, varicosities were seen in the most distal portions of the neurites, away from the cell body, as has been described in the early stages of Wallerian-like degeneration of axonal processes (Fayaz and Tator, 2000). At higher doses of ddC, these effects were more prominent, and frank neuritic degeneration was also seen. ddl and d4T had similar effects to ddC, but required higher doses for an effect similar to that of ddC. AZT, on the other hand, did not have any appreciable effect on neuritic morphology, even at high doses. FIG. 1 (F and G) shows dose-dependent reduction by ddC, ddl and D4T, but not by AZT, of total neuritic length and number of neurites per neuron. These results parallel the findings in HIV-infected individuals; AZT does not cause a neuropathy and ddC has the highest incidence of neuropathy, followed by ddl and d4T (Moore et al., 2000; Moyle and Sadler, 1998).

### EXAMPLE 2

### FK506 Prevents the Development of ddC-Induced Neurotoxicity in Primary DRG Neurons

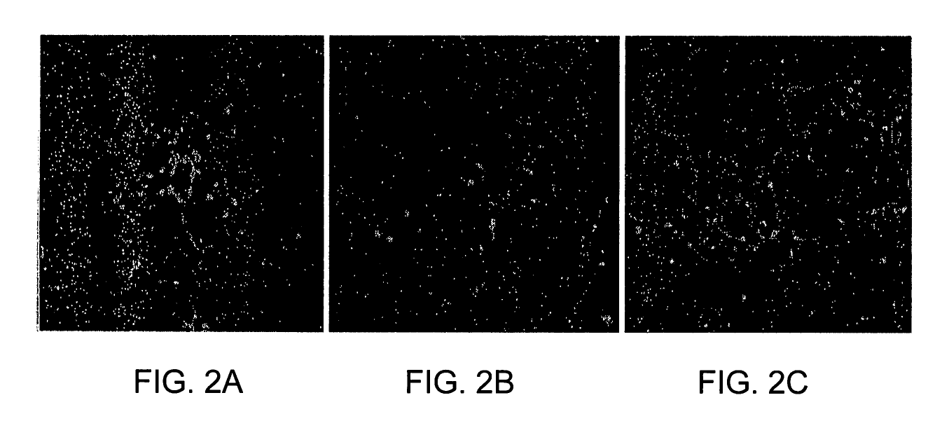

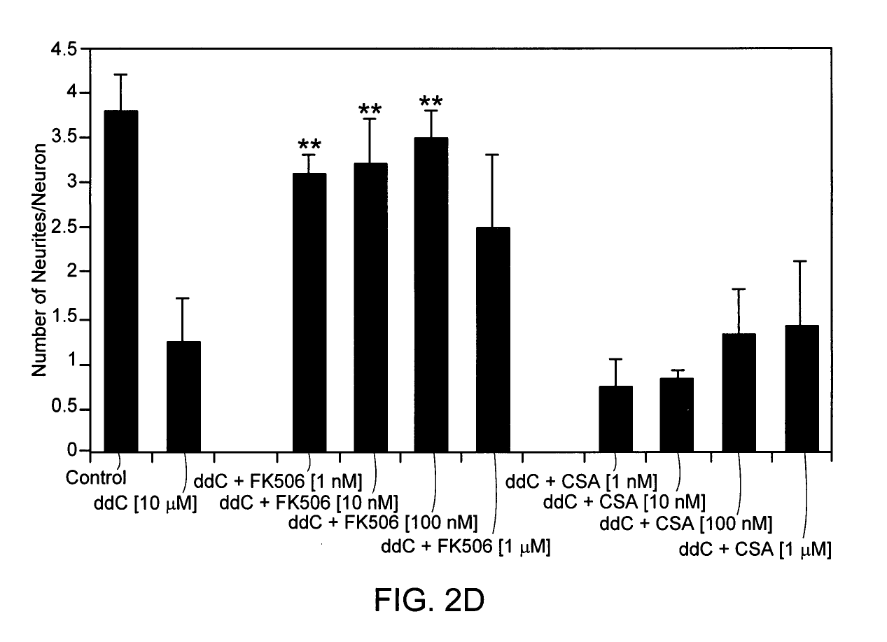

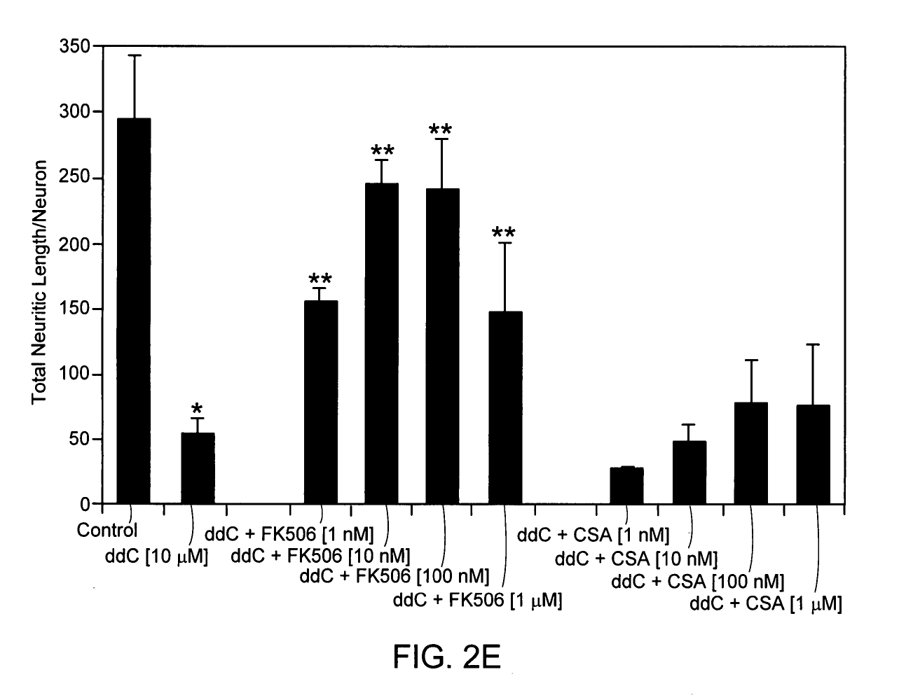

To test whether FK506 may prevent NRTI neurotoxicity in primary DRG sensory neurons, ss before, neurons were plated for 3 hours for the establishment of neurites. Then added ddC (10 μM) was added with or without FK506 and morphological changes were analyzed 15 hours later. As seen in FIG. 2 very low doses of FK506 prevented the reduction in number of neurites per neuron and total neuritic length by ddC. In contrast to FK506, CSA was not effective in preventing this neurotoxicity. In the absence of ddC, FK506 did not have any appreciable effect on the morphological parameters used in the study.

### EXAMPLE 3

### NRTI Neurotoxicity is Associated with Loss of Δ-σ in Neuronal Mitochondria Four Hours after Exposure

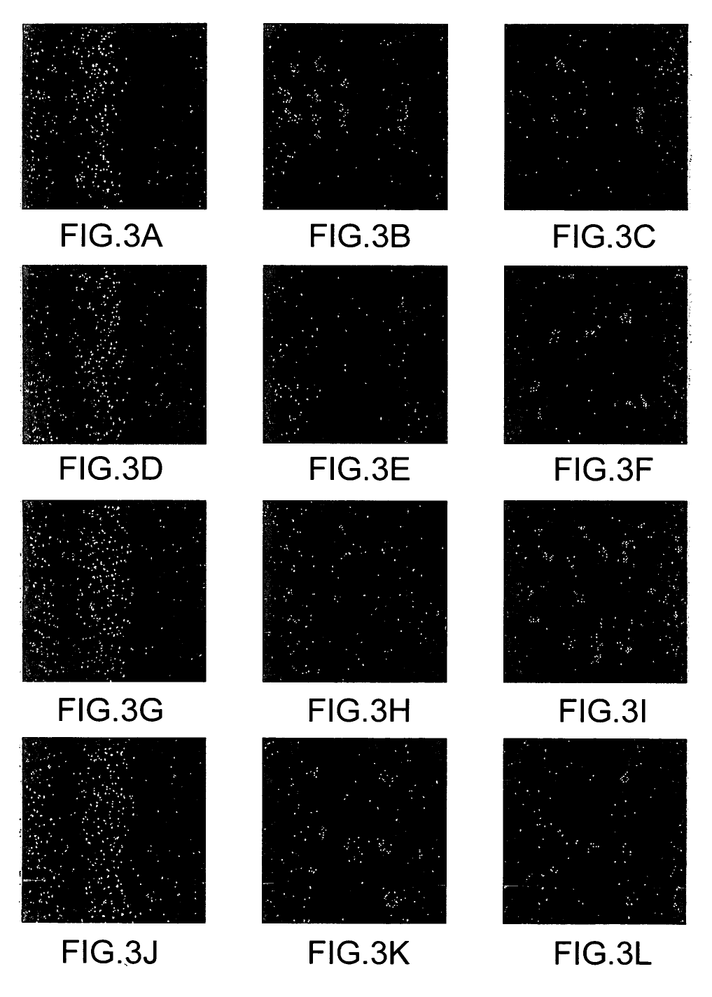

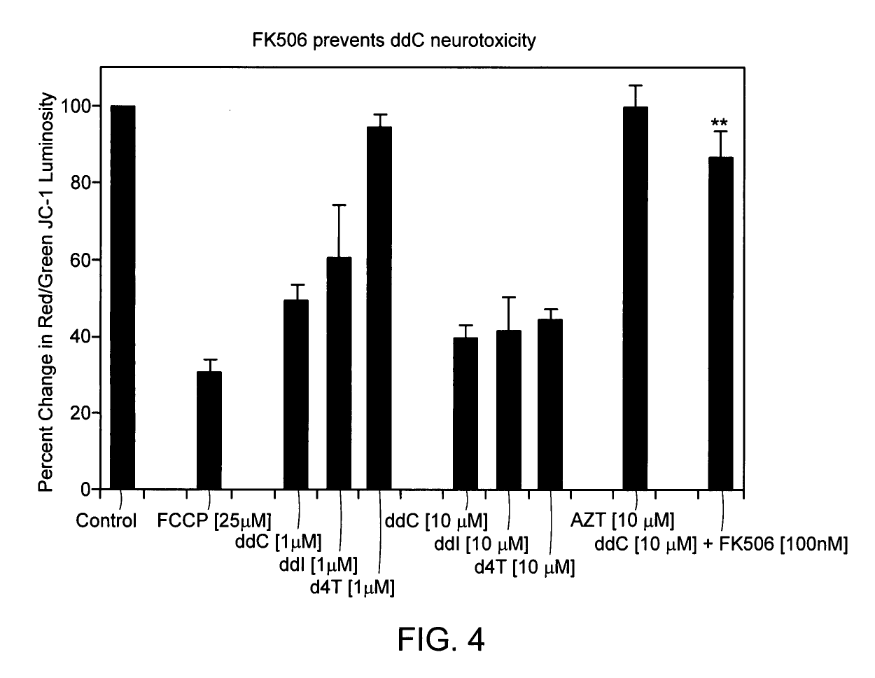

JC-1 is a lipophilic, cationic dye that emits green fluorescence at low concentrations when it is in monomeric form but emits a red fluorescence when aggregated. In healthy mitochondria with an intact membrane potential differential (Δ-σ), the fluorescence emission pattern of JC-1 exhibits a high red: green luminosity ratio, while in depolarized mitochondria, this ratio is low. Tetanus Toxin C-fragment was used as a live neuronal marker in our mixed DRG neuronal/Schwann cell cultures, and FCCP, a protonophore and uncoupler of oxidative phosphorylation, served as a positive control since it is a prototypic depolarizer of mitochondrial membrane. In vehicle control cultures, TTC-labeled neurons emitted mainly red and little green fluorescence (FIG. 3). The reverse was true for both the ddC- and FCCP-treated cultures, suggesting loss of mitochondrial membrane potential. FIG. 4 graphically shows the JC-1 red:green luminosity ratios for DRG cultures treated with 4 hours of varying doses of ddC, ddl, d4T and AZT, as well as the ratios for the vehicle control cultures and FCCP-treated cultures. Dose-dependent reduction of JC-1 red: green luminosity ratio was observed for ddC, ddl and d4T, with ddC being the most potent and d4T being the least. (1 μM) ddC, (1 μM) ddl and (10 μM) d4T achieved the same level of mitochondrial membrane depolarization as (25 μM) FCCP. In contrast, AZT did not cause significant reduction in this ratio, compared to control cultures. Of note, there was no evidence of mitochondrial depolarization in ddC-treated Schwann cells.

### EXAMPLE 4

### FK506 Prevents Loss of Δ-σ in Neuronal Mitochondria Associated with NRTI Exposure

The results of experiments with JC-1 measurement in neuronal mitochondria with FK-506 application paralleled the morphological outcome measures of neurotoxicity. When FK506 was applied at the time of exposure to ddC, a similar reduction was not seen in membrane potential differential in neuronal mitochondria (FIG. 3 J-L and FIG. 4). These results suggest that FK506 was able to prevent toxicity of ddC on mitochondria within neurons:

### EXAMPLE 5

### Exposure to ddC Causes Neuronal Cell Death, Preventable by FK506

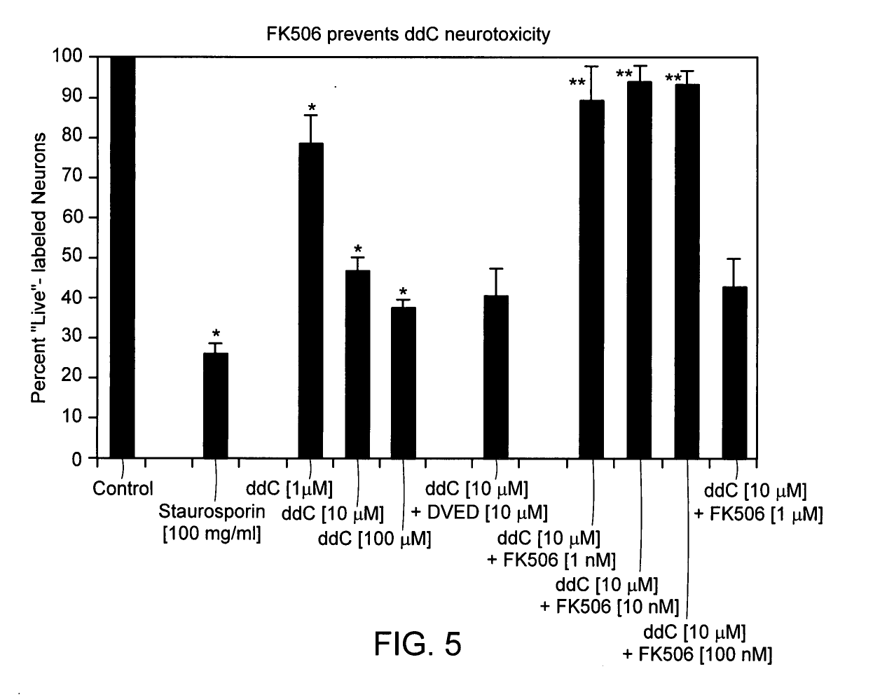

Since toxicity occurred on established neuritic processes and neuronal mitochondria, it was investigated if there was any cell death from exposure to NRTIs. It was found that a subpopulation of neurons was susceptible to death by ddC exposure for 18 hours and exhibited positive “dead-cell” staining using a Live/Death kit (Molecular Probes) because of its inability to exclude ethidium homodimer. This occurred in a dose-dependent manner and higher doses of ddC were associated with a lower percentage of neurons labeling with the “live-cell” marker (FIG. 5). This cell death was thought to be necrotic rather than apoptotic on the basis of increased size of the dead cells and lack of DNA laddering in neurons on TUNEL staining; a marker of apoptosis (data not shown). Furthermore, DEVD, a specific caspase 3 inhibitor, did not prevent the cell death (FIG. 5). Of note, there was no cell death in ddC-treated Schwann cells, even at a ddC dose of 1 mM (data not shown).

When FK506 was added to the culture media at the time of ddC addition, there was a remarkable lack of positive “dead-cell” staining at 18 hours (FIG. 5). This prevention of neuronal cell death was most prominent at the lower concentrations of FK506 (10-100 nM). In contrast to FK506, CSA did not prevent neuronal cell death by ddC (data not shown).

All publications and patent applications disclosed herein are incorporated into this application by reference in their entirety, including U.S. Pat. Nos. 6,362,160; 5,898,029; 6,080,753; 5696,135; 3,060,373; 5,798,355 and PCT/US96/17677 and WO96/40140 disclose generic and specific immunophilins and neuroimmunophilins, which can be used in the instant invention.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed and equivalent within the spirit of the invention as defined by the scope of the claims.

### REFERENCES

Ankarcrona M, Dypbukt J M, Bonfoco E, Zhivotovsky B, Orrenius S, Lipton S A, et al. Glutamate-induced neuronal death: a succession of necrosis or apoptosis depending on mitochondrial function. Neuron 1995; 15: 961-73.

Ankarcrona M, Dypbukt J M, Orrenius S, Nicotera P. Calcineurin and mitochondrial function in glutamate-induced neuronal cell death. FEBS Left 1996; 394: 321-4.

Barile M, Valenti D, Hobbs G A, Abruzzese M F, Keilbaugh S A, Passarella S, et al. Mechanisms of toxicity of 3′-azido-3′-deoxythymidine. Its interaction with adenylate kinase. Biochem Pharmacol 1994; 48: 1405-12.

Barile M, Valenti D; Passarella S, Quagliariello E. 3′-Azido-3′-deoxythmidine uptake into isolated rat liver mitochondria and impairment of ADP/ATP translocator. Biochem Pharmacol 1997; 53: 913-20.

Benbrik E, Chariot P, Bonavaud S, Ammi-Said M, Frisdal E, Rey C, et al. Cellular and mitochondrial toxicity of zidovudine (AZT), didanosine (ddl) and zalcitabine (ddC) on cultured human muscle cells. J Neurol Sci 1997; 149: 19-25.

Brew B, Tisch S, Law M. Lactate concentrations distinguish between nucleoside neuropathy and HIV distal symmetrical sensory polyneuropathy. 8th Conference on Retrovirus and Opportunistic Infections. Chicago, 2001: abstract 9.

Brockes J P, Fields K L, Raff M C. Studies on cultured rat Schwann cells. I. Establishment of purified populations from cultures of peripheral nerve. Brain Res 1979; 165: 105-18.

Chen C H, Cheng Y C. Delayed cytotoxicity and selective loss of mitochondrial DNA in cells treated with the anti-human immunodeficiency virus compound 2′,3′-dideoxycytidine. J Biol Chem 1989; 264: 11934-7.

Chen C H, Vazquez-Padua M, Cheng Y C. Effect of anti-human immunodeficiency virus nucleoside analogs on mitochondrial DNA and its implication for delayed toxicity. Mol Pharmacol 1991; 39: 625-8.

Cherry C L, Gahan M E, McArthur J C, Lewin S R, Hoy J F, Wesselingh S L. Exposure to dideoxynucleosides is reflected in lowered mitochondrial DNA in subcutaneous fat. J AIDS 2002 In press.

Cossarizza A, Baccarani-Contri M, Kalashnikova G, Franceschi C. A new method for the cytofluorimetric analysis of mitochondrial membrane potential using the J-aggregate forming lipophilic cation 5,5′,6,6′-tetrachloro-1,1′,3,3′-tetraethylbenzimidazolcarbocyanine iodide (JC-1). Biochem Biophys Res Commun 1993; 197: 40-5.

Costantini L C, Chaturvedi P, Armistead D M, McCaffrey P G, Deacon T W, Isacson O. A novel immunophilin ligand: distinct branching effects on dopaminergic neurons in culture and neurotrophic actions after oral administration in an animal model of Parkinson's disease. Neurobiol Dis 1998; 5: 97-106.

Crompton M, Virji S, Ward J M. Cyclophilin-D binds strongly to complexes of the voltage-dependent anion channel and the adenine nucleotide translocase to form the permeability transition pore. Eur J Biochem 1998; 258: 729-35.

Cui L, Locatelli L, Xie M Y, Sommadossi J P. Effect of nucleoside analogs on neurite regeneration and mitochondrial DNA synthesis in PC-12 cells. J Pharmacol Exp Ther 1997; 280: 1228-34.

Davis G E, Manthorpe M, Varon S. Parameters of neuritic growth from ciliary ganglion neurons in vitro: influence of laminin, schwannoma polyomithine-binding neurite promoting factor and ciliary neuronotrophic factor. Brain Res 1985; 349: 75-84.

Dawson T M, Steiner J P, Dawson V L, Dinerman J L, Uhl G R, Snyder S H. Immunosuppressant FK506 enhances phosphorylation of nitric oxide synthase and protects against glutamate neurotoxicity. Proc Natl Acad Sci U S A 1993; 90: 9808-12.

Dubinsky J M, Levi Y. Calcium-induced activation of the mitochondrial permeability transition in hippocampal neurons. J Neurosci Res 1998; 53: 728-41.

Eldridge C F, Bunge M B, Bunge R P, Wood P M. Differentiation of axon-related Schwann cells in vitro. I. Ascorbic acid regulates basal lamina assembly and myelin formation. J Cell Biol 1987; 105: 1023-34.

Famularo G, Moretti S, Marcellini S, Trinchieri V, Tzantzoglou S, Santini G, et al. Acetyl-carnitine deficiency in AIDS patients with neurotoxicity on treatment with antiretroviral nucleoside analogues. Aids 1997; 11: 185-90.

Fayaz I, Tator C H. Modeling axonal injury in vitro: injury and regeneration following acute neuritic trauma. J Neurosci Methods 2000; 102: 69-79.

Gold B G. Neuroimmunophilin ligands: evaluation of their therapeutic potential for the treatment of neurological disorders. Expert Opin Investig Drugs 2000; 9: 2331-42.

Hobbs G A, Keilbaugh S A, Rief P M, Simpson M V. Cellular targets of 3′-azido-3′-deoxythymidine: an early (non-delayed) effect on oxidative phosphorylation. Biochem Pharmacol 1995; 50: 381-90.

Jiang S, Cai J, Wallace D C, Jones D P. Cytochrome c-mediated apoptosis in cells lacking mitochondrial DNA. Signaling pathway involving release and caspase 3 activation is conserved. J Biol Chem 1999; 274: 29905-11.

Kaibori M, Inoue T, Tu W, Oda M, Kwon A H, Kamiyama Y, et al. FK506, but not cyclosporin A, prevents mitochondrial dysfinction during hypoxia in rat hepatocytes. Life Sci 2001; 69: 17-26.

Lazarovici P, Yanai P, Yavin E. Molecular interactions between micellar polysialogangliosides and affinity-purified tetanotoxins in aqueous solution. J Biol Chem 1987; 262: 2645-51.

Lewis L D, Hamzeh F M, Lietman P S. Ultrastructural changes associated with reduced mitochondrial DNA and impaired mitochondrial function in the presence of 2′3′-dideoxycytidine. Antimicrob Agents Chemother 1992; 36: 2061-5.

Lewis W, Dalakas M C. Mitochondrial toxicity of antiviral drugs. Nat Med 1995; 1: 417-22.

Mathur A, Hong Y, Kemp B K, Barrientos A A, Erusalimsky J D. Evaluation of fluorescent dyes for the detection of mitochondrial membrane potential changes in cultured cardiomyocytes [see comments]. Cardiovasc Res 2000; 46: 126-38.

Modica-Napolitano J S. AZT causes tissue-specific inhibition of mitochondrial bioenergetic function. Biochem Biophys Res Commun 1993; 194: 170-7.

Moore R D, Wong W M, Keruly J C, McArthur J C. Incidence of neuropathy in HIV-infected patients on monotherapy versus those on combination therapy with didanosine, stavudine and hydroxyurea. Aids 2000; 14: 273-8:

Moyle G J, Sadler M. Peripheral neuropathy with nucleoside antiretrovirals: risk factors, incidence and management. Drug Saf 1998; 19: 481-94.

Reers M, Smith T W, Chen L B. J-aggregate formation of a carbocyanine as a quantitative fluorescent indicator of membrane potential. Biochemistry 1991; 30: 4480-6.

Schweizer M, Schlegel J, Baumgartner D, Richter C. Sensitivity of mitochondrial peptidyl-prolyl cis-trans isomerase, pyridine nucleotide hydrolysis and Ca2+ release to cyclosporine A and related compounds. Biochem Pharmacol 1993; 45: 641-6.

Sezen S F, Hoke A, Burnett A L, Snyder S H. Immunophilin ligand FK506 is neuroprotective for penile innervation. Nat Med 2001; 7: 1073-4.

Sharkey J, Butcher S P. Immunophilins mediate the neuroprotective effects of FK506 in focal cerebral ischaemia. Nature 1994; 371: 336-9.

Skuta G, Fischer G M, Janaky T, Kele Z, Szabo P, Tozser J, et al. Molecular mechanism of the short-term cardiotoxicity caused by 2′,3′-dideoxycytidine (ddC): modulation of reactive oxygen species levels and ADP-ribosylation reactions. Biochem Pharmacol 1999; 58: 1915-25.

Snyder S H, Sabatini D M, Lai M M, Steiner J P, Hamilton G S, Suzdak P D. Neural actions of immunophilin ligands. Trends Pharmacol Sci 1998; 19: 21-6.

Starnes M C, Cheng Y C. Cellular metabolism of 2′,3′-dideoxycytidine, a compound active against human immunodeficiency virus in vitro. J Biol Chem 1987; 262: 988-91.

Steiner J P, Connolly M A, Valentine H L, Hamilton G S, Dawson T M, Hester L, et al. Neurotrophic actions of nonimmunosuppressive analogues of immunosuppressive drugs FK506, rapamycin and cyclosporin A. Nat Med 1997; 3: 421-8.

Stockel K, Schwab M, Thoenen H. Comparison between the retrograde axonal transport of nerve growth factor and tetanus toxin in motor, sensory and adrenergic neurons. Brain Res 1975; 99: 1-16.

Toung T J, Bhardwaj A, Dawson V L, Dawson T M, Traystman R J, Hum P D. Neuroprotective FK506 does not alter in vivo nitric oxide production during ischemia and early reperfusion in rats. Stroke 1999; 30: 1279-85.

Wang H G, Pathan N, Ethell I M, Krajewski S, Yamaguchi Y, Shibasaki F, et al. Ca2+-induced apoptosis through calcineurin dephosphorylation of BAD. Science 1999; 284: 339-43.

White R J, Reynolds I J. Mitochondrial depolarization in glutamate-stimulated neurons: an early signal specific to excitotoxin exposure. J Neurosci 1996; 16: 5688-97. Wulff E A, Wang A K, Simpson D M. HIV-associated peripheral neuropathy: epidemiology, pathophysiology and treatment. Drugs 2000; 59: 1251-60.

1. A method of identifying immunophilin ligands useful for the treatment of antiretroviral toxic neuropathies comprising the determination of the immunophilin ligands effect on NRTI induced mitochondrial toxicity in neuronal cells.

2. A method of identifying immunophilin ligands useful for the treatment of antiretroviral toxic neuropathies comprising;
a) treating separate neuronal cell cultures with a NRTI, the same NRTI and an immunophilin ligand and vehicle control;
b) determining the mitochondrial toxicity in the separately treated cultures;
c) comparing the results on mitochondrial toxicity in the separately treated cultures;
d) identifying an immunophilin ligand which results in less mitochondrial toxicity due to the treatment with NRTI.

3. A method of identifying immunophilin ligands useful for the treatment of antiretroviral toxic neuropathies comprising;
a) treating separate neuronal cell cultures with a NRTI, the same NRTI and an immunophilin ligand and vehicle control;
b) determining the effects on the number of neurites and total neuritic length per neuron in the separately treated cultures;
c) comparing the results on the number of neurites and total neuritic length per neuron in the separately treated cultures;
d) identifying an immunophilin ligand which results in preventing reduction in the number of neurites and total neuritic length per neuron due to the treatment with NRTI.

4. A method of identifying immunophilin ligands useful for the treatment of antiretroviral toxic neuropathies comprising;
a) treating separate neuronal cell cultures with a NRTI, the same NRTI and an immunophilin ligand and vehicle control;
b) determining the effects on neuronal cell death in the separately treated cultures;
c) comparing the results on neuronal cell death in the separately treated cultures;
d) identifying an immunophilin ligand which results in preventing neuronal cell death due to the treatment with NRTI.

5. The methods of claims 1-4, wherein the immunophilin ligand is non-immunosuppressive.

6. The methods of claims 1-4, wherein the neuronal cell culture comprises dorsal root ganglion sensory neurons.

7. The methods of claims 1-4, wherein the NRTI is ddC.

8. The methods of claims 1-4, wherein the NRTI is ddI.

9. The methods of claims 1-4, wherein the NRTI is d4T.

10. The methods of claims 1-5, wherein the immunophilin ligand is FK506 analog.

11. The method of claim 10, wherein the FK506 analog is 3-(3-pyridyl)-1-propyl (2S)-1-(3,3-dimethyl-1,2-dioxopentyl)-2-pyrrolidinedinecarboxylate .

12. A method for the treatment of antitretroviral toxic neuropathy comprising the administration to a patient in need of such treatment a non-immunosuppressive immunophilin ligand.

13. A method of claim 12, wherein the non-immunosuppressive ligand is a FK506 analog.

14. A method of claim 13, wherein the FK506 analog is 3-(3-pyridyl)-1-propyl (2S)-1-(3,3-dimethyl-1,2-dioxopentyl)-2-pyrrolidinedinecarboxylate.

15. A method for the prevention of antitretroviral toxic neuropathy comprising the administration to a patient in need of such prevention a non-immunosuppressive immunophilin ligand.

16. A method of claim 15, wherein the non-immunosuppressive ligand is a FK506 analog.

17. A method of claim 16, wherein the FK506 analog is 3-(3-pyridyl)-1-propyl (2S)-1-(3,3-dimethyl-1,2-dioxopentyl)-2-pyrrolidinedinecarboxylate.
